(19) United States
(12) Patent Application Publication
(10) Pub. No.: US 2007/0135403 A1
(43) Pub. Date: Jun. 14, 2007

(54) Substituted 2,5-diaminomethyl-1H-pyrrole compounds

(75) Inventors: Beatrix Merla, Aachen (DE); Corinna Sundermann, Aachen (DE); Utz-Peter Jagusch, Aachen (DE); Werner Englberger, Stolberg (DE); Hagen-Heinrich Hennies, Simmerath (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/600,876

(22) Filed: Nov. 17, 2006

Related U.S. Application Data
(63) Continuation of application No. PCT/EP05/05402, filed on May 17, 2005.

(30) Foreign Application Priority Data
May 17, 2004 (DE) 10 2004 024 773.0

(51) Int. Cl. A61K 31/55; A61K 31/541; A61K 31/5377; A61K 31/496; A61K 31/4025; C07D 417/02; C07D 413/02; C07D 403/02

(52) U.S. Cl. 514/210.2; 514/217.08; 514/235.2; 514/227.5; 514/254.01; 514/326; 514/422; 544/60; 544/372; 546/205; 548/517; 548/518; 540/602; 540/480; 544/141

(57) ABSTRACT

Substituted 2,5-diaminomethyl-1H-pyrroles, a process for the production thereof, pharmaceutical compositions containing them, and the use of these compounds for regulating 5-hydroxytryptamine uptake, noradrenalin uptake and/or opioid receptor activity, or for treating or inhibiting disorders or diseases at least partially mediated by a receptor selected from the group consisting of 5-hydroxytryptamine receptors, noradrenalin receptors and opioid receptors.

### CROSS REFERENCE TO RELATED APPLICATIONS

This application is a continuation of international application no. PCT/EP2005/005402, filed May 17, 2005 designating the United States of America and published in German on Dec. 1, 2005 as WO 2005/113549, the entire disclosure of which is incorporated herein by reference. Priority is claimed based on Federal Republic of Germany patent application no. DE 10 2004 024 773.0, filed May 17, 2004.

### BACKGROUND OF THE INVENTION

The present invention relates to substituted 2,5-diaminomethyl-1H-pyrroles, to a process for the production thereof, to pharmaceutical preparations containing these compounds and to the use of these compounds for the production of pharmaceutical preparations.

Pain is one of the basic clinical symptoms. There is a worldwide need for effective pain treatments. The urgency of the requirement for therapeutic methods for providing tailored and targeted treatment of chronic and non-chronic pain, this being taken to mean pain treatment which is effective and satisfactory from the patient's standpoint, is evident from the large number of scientific papers relating to applied analgesia and to basic nociception research which have appeared in recent times.

Conventional opioids, such as for example morphine, are effective in the treatment of severe to very severe pain, but they exhibit unwanted accompanying symptoms, such as for example respiratory depression, vomiting, sedation or constipation. Research is being carried out worldwide into other pain-relieving agents.

### SUMMARY OF THE INVENTION

An object of the present invention was accordingly to provide new pharmacologically active compounds.

Another object of the invention was to provide new compounds which are useful for treating, in particular, pain.

These and other objects have been achieved in accordance with the present invention by providing the substituted 2,5-diaminomethyl-1H-pyrroles corresponding to the following formula I.

It has surprisingly been found that the substituted 2,5-diaminomethyl-1H-pyrroles according to the invention of formula I below exhibit elevated affinity for opioid receptors, in particular for μ opioid receptors, and are accordingly suitable for regulating these receptors.

The substituted 2,5-diaminomethyl-1H-pyrroles according to the invention of formula I are furthermore suitable for regulating, preferably for inhibiting noradrenalin (NA) uptake and for regulating, preferably for inhibiting, 5-hydroxytryptamine (5-HT) uptake.

The substituted 2,5-diaminomethyl-1H-pyrrole compounds according to the invention of formula I below may accordingly in particular be used as pharmacological active ingredients in pharmaceutical preparations for the prevention and/or treatment of disorders and diseases associated with the above-stated receptors or processes.

The present invention accordingly provides substituted 2,5-diaminomethyl-1H-pyrroles corresponding to the following formula I: 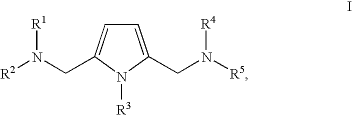
in which
R¹ and R² together with the nitrogen atom to which they are attached form a saturated or unsaturated cycloaliphatic residue optionally comprising at least one further heteroatom as a ring member, which cycloaliphatic residue may be identically or differently mono- or polysubstituted with a substituent selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl;
R³ denotes a linear or branched, unsaturated or saturated, unsubstituted or at least monosubstituted aliphatic residue,
R⁴ denotes hydrogen, a linear or branched, unsubstituted or at least monosubstituted, unsaturated or saturated aliphatic residue, an unsaturated or saturated, unsubstituted or at least monosubstituted, cycloaliphatic residue optionally comprising at least one heteroatom as a ring member, which cycloaliphatic residue may be attached via a linear or branched alkylene group, or an unsubstituted or at least monosubstituted aryl residue attached via a linear or branched alkylene group,
R⁵ denotes a linear or branched, unsubstituted or at least monosubstituted, unsaturated or saturated aliphatic residue, an unsaturated or saturated, unsubstituted or at least monosubstituted cycloaliphatic residue optionally comprising at least one heteroatom as a ring member, which cycloaliphatic residue may be attached via a linear or branched alkylene group, or an unsubstituted or at least monosubstituted aryl residue attached via a linear or branched alkylene group, or
R⁴ and R⁵ together with the nitrogen atom to which they are attached form a saturated or unsaturated , cycloaliphatic residue optionally comprising at least one further heteroatom as a ring member, which cycloaliphatic residue may be identically or differently mono- or polysubstituted with a substituent selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —NH₂, —NH-C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

The compounds of formula I in which residues R¹ and R² and residues R⁴ and R⁵ in each case together with the nitrogen atom to which they are attached form the same residue selected from the group consisting of pyrrolidinyl, morpholinyl and piperidinyl, and residue R³ in each case denotes a methyl group, i.e. the compounds 2,5-bis(N-piperidinyl-methyl)-1-methylpyrrole, 2,5-bis(N-morpholinyl-methyl)-1-methylpyrrole and 2,5-bis(N-pyrrolidinyl-methyl)-1-methylpyrrole, and compounds of formula I, in which residues R¹ and R² together with the nitrogen atom joining them together as a ring member form a piperidinyl residue and residues R³ to R⁵ in each case denote a methyl group, are preferably excepted.

Compounds of formula I, in which residues R¹ and R² and residues R⁴ and R⁵ in each case together with the nitrogen atom joining them together as a ring member form the same residue selected from the group consisting of pyrrolidinyl, morpholinyl and piperidinyl, and residue R³ in each case denotes a methyl group, i.e. the compounds 2,5-bis(N-piperidinyl-methyl)1-methylpyrrole, 2,5-bis(N-morpholinyl-methyl)-1-methylpyrrole and 2,5-bis(N-pyrrolidinyl-methyl)-1-methylpyrrole, and compounds of formula I, in which residues R¹ and R² together with the nitrogen atom joining them together as a ring member form a piperidinyl residue and residues R³ to R⁵ in each case denote a methyl group, and optionally in each case the corresponding salts thereof may likewise preferably be excepted.

Preferred substituted 2,5-diaminomethyl-1H-pyrroles according to the invention of formula I are those in which residues R¹ and R² together with the nitrogen atom joining them together as a ring member form a saturated or unsaturated 4-, 5, 6-, 7-, 8- or 9-membered cycloaliphatic residue optionally comprising at least one further heteroatom as a ring member, which cycloaliphatic residue may be identically or differently mono- or polysubstituted with a substituent selected from the group consisting of C₁₋₅ alkyl, —C(═O)—O—C₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

R¹ and R² together with the nitrogen atom joining them together as a ring member preferably form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which cycloaliphatic residue may be identically or differently mono- or polysubstituted with a substituent selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

R¹ and R² together with the nitrogen atom joining them together as a ring member particularly preferably form an imidazolidinyl, aziridinyl, azetidinyl, pyrrolidinyl, piperidinyl, azepanyl, azocanyl, piperazinyl, morpholinyl or thiomorpholinyl residue, which residue may be identically or differently mono- or polysubstituted with a substituent selected from the group consisting of C₁₋₅ alkyl, —C(═O)—O—C₁₋₅-alkyl, O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl and residues R³ to R⁵ in each case have the above-stated meaning, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

Further preferred substituted 2,5-diaminomethyl-1H-pyrroles according to the invention of formula I are those in which residue R³ denotes a linear or branched, saturated or unsaturated, unsubstituted or at least monosubstituted aliphatic C₁₋₁₀ residue. R³ preferably denotes a linear or branched, unsubstituted or at least monosubstituted C₁₋₅ alkyl residue. R³ particularly preferably denotes an alkyl residue selected from the group consisting of methyl, ethyl, n-propyl, iso-propyl, n-butyl, iso-butyl, sec-butyl and tert-butyl and residues R¹, R², R⁴ and R⁵ in each case have the above-stated meaning, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

Substituted 2,5-diaminomethyl-1H-pyrroles according to the invention of formula I are furthermore those in which residue R⁴ denotes hydrogen, a linear or branched, saturated or unsaturated, unsubstituted or at least monosubstituted aliphatic C₁₋₁₀ residue, a saturated or unsaturated, unsubstituted or at least monosubstituted 3-, 4-, 5-, 6-, 7-, 8- or 9-membered cycloaliphatic residue optionally comprising at least one heteroatom as a ring member, which cycloaliphatic residue may be attached via a linear or branched C₁₋₃ alkylene group, or an unsubstituted or at least monosubstituted 5- to 14-membered aryl residue attached via a linear or branched C₁₋₃ alkylene group. R⁴ preferably denotes hydrogen, a linear or branched C₁₋₅ alkyl residue, a saturated or unsaturated 4-, 5-, 6-, 7-, 8- or 9-membered cycloaliphatic residue, which may be attached via a linear or branched C₁₋₃ alkylene group, or an unsubstituted or at least monosubstituted phenyl residue attached via a linear or branched C₁₋₃ alkylene group. R⁴ particularly preferably denotes hydrogen residue, an alkyl residue selected from the group consisting of methyl, ethyl, n-propyl, iso-propyl, n-butyl, iso-butyl, sec-butyl and tert-butyl or a benzyl residue and residues R¹ to R³, R⁵ and R⁴ and R⁵ together in each case have the above-stated meaning, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

Further preferred substituted 2,5-diaminomethyl-1H-pyrroles according to the invention of formula I are those in which residue R⁵ denotes a linear or branched, saturated or unsaturated, unsubstituted or at least monosubstituted aliphatic C₁₋₁₀ residue, a saturated or unsaturated, unsubstituted or at least monosubstituted 3-, 4-, 5-, 6-, 7-, 8- or 9-membered cycloaliphatic residue optionally comprising at least one heteroatom as a ring member, which cycloaliphatic residue may be attached via a linear or branched C₁₋₃ alkylene group, or denotes an unsubstituted or at least monosubstituted 5- to 14-membered aryl residue attached via a linear or branched C₁₋₃ alkylene group. R⁵ preferably denotes a linear or branched C₁₋₅ alkyl residue, a saturated or unsaturated 4-, 5-, 6-, 7-, 8- or 9-membered cycloaliphatic residue, which may be attached via a linear or branched C₁₋₃ alkylene group, or denotes an unsubstituted or at least monosubstituted phenyl residue attached via a linear or branched C₁₋₃ alkylene group. R⁵ particularly preferably denotes an alkyl residue selected from the group consisting of methyl, ethyl, n-propyl, iso-propyl, n-butyl, iso-butyl, sec-butyl and tert-butyl or a benzyl residue and residues R¹ to R⁴ and R⁴ and R⁵ together in each case have the above-stated meaning, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

Preferred substituted 2,5-diaminomethyl-1H-pyrroles according to the invention of formula I are furthermore those in which residues R⁴ and R⁵ together with the nitrogen atom joining them together as a ring member form a saturated or unsaturated 4-, 5-, 6-, 7-, 8- or 9-membered cycloaliphatic residue optionally comprising a heteroatom as a ring member, which cycloaliphatic residue may be identically or differently mono- or polysubstituted with a substituent selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₅-alkyl, —C(═O)—N(C₁₋₅-alkyl)₂, —C(═O)—C₁₋₅-alkyl, —NH₂, —NH—C₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

R⁴ and R⁵ together with the nitrogen atom joining them together as a ring member preferably form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which cycloaliphatic residue may be identically or differently mono- or polysubstituted with a substituent selected from the group consisting of C₁₋₅ alkyl, —C(═O)—O—C₁₋₅-alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

R⁴ and R⁵ together with the nitrogen atom joining them together as a ring member particularly preferably form an imidazolidinyl, aziridinyl, azetidinyl, pyrrolidinyl, piperidinyl, azepanyl, azocanyl, piperazinyl, morpholinyl or thiomorpholinyl residue, which residue may be identically or differently mono- or polysubstituted with a substituent selected from the group consisting of C₁₋₅-alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, and residues R¹ to R³ and R⁴ and R⁵ separately from one another in each case have the above-stated meaning, in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

If one or more residues R³ to R⁵ denote a linear or branched, saturated or unsaturated aliphatic residue, i.e. a linear or branched alkyl, alkenyl or alkynyl residue, which may be mono- or polysubstituted, for example mono-, di-, tri-, tetra or pentasubstituted, these substituents may mutually independently preferably be selected from the group consisting of halogen, hydroxy, —CN, —CF₃, —CHF₂, —CH₂F, C₁₋₆ alkoxy and optionally at least monosubstituted phenyl, particularly preferably from the group consisting of F, Cl, Br and hydroxy. According to the invention, the aliphatic residues may only comprise such substituents which are attached via a single bond, i.e. polyvalent substituents such as for example an oxo-group (═O) are not included. If the above-stated phenyl substituent is itself mono- or polysubstituted, for example mono-, di-, tri-, tetra- or pentasubstituted, the substituents thereof may in each case mutually independently preferably be selected from the group consisting of hydroxy, F, Cl, Br, I, —NH₂, —NH—C₁₋₅-alkyl, —N(C₁₋₅-alkyl)₂, —C(═O)—NH₂, —C(═O)—NH—C₁₋₅-alkyl, —C(═O)—N(C₁₋₅-alkyl)₂, —NO₂, —CN, —CF₃, —CHF₂, —CH₂F, C₁₋₅ alkyl and C₁₋₅ alkoxy.

Examples of suitable alkyl, alkenyl and alkynyl residues, which may be mono- or polysubstituted, for example mono-, di-, tri-, tetra- or pentasubstituted, include methyl, ethyl, n-propyl, iso-propyl, n-butyl, iso-butyl, sec-butyl, tert-butyl, n-pentyl, iso-pentyl, neopentyl, n-hexyl, 1-hexyl, n-heptyl, n-octyl, n-nonyl, n-decyl, —C(H)(C₂H₅)₂, —C(H)(n-C₃H₇)₂, —CH₂—CH₂—C(H)(CH₃)—CH₂)₃—CH₃, vinyl, ethynyl, propenyl, allyl, propynyl, butenyl, butynyl, pentenyl, pentynyl, hexenyl, hexynyl.

The monocyclic cycloaliphatic residue optionally comprising at least one further heteroatom as a ring member formed by R¹ and R² together with the nitrogen atom joining them together as a ring member is saturated or unsaturated, but not aromatic. It may be identically or differently mono- or polysubstituted, for example mono-, di-, tri-, tetra- or pentasubstituted, wherein the particular substituents may mutually independently preferably be selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

The particular furanyl, thiophenyl, pyridinyl, phenyl or benzyl substituent may itself be mono- or polysubstituted, optionally mono-, di-, tri-, tetra- or pentasubstituted, wherein the substituents thereof may mutually independently preferably be selected from the group consisting of hydroxy, F, Cl, Br, I, —NH₂, —NH—C₁₋₅-alkyl, —N(C₁₋₅-alkyl)₂, —C(═O)—NH₂, —C(═O)—NH-C₁₋₅-alkyl, —C(═O)—N(C₁₋₅-alkyl)₂, —NO₂, —CN, —CF₃, —CHF₂, —CH₂F, C₁₋₅-alkyl and C₁₋₅-alkoxy.

The monocyclic cycloaliphatic residue optionally comprising at least one further heteroatom as a ring member formed by R⁴ and R⁵ together with the nitrogen atom joining them together as a ring member is saturated or unsaturated, but not aromatic. It may be identically or differently mono- or polysubstituted, for example mono-, di-, tri-, tetra- or pentasubstituted, wherein the substituents may mutually independently preferably be selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

The particular furanyl, thiophenyl, pyridinyl, phenyl or benzyl substituent may itself be unsubstituted or mono- or polysubstituted, optionally mono-, di-, tri-, tetra- or pentasubstituted, wherein the substituents thereof may mutually independently preferably be selected from the group consisting of hydroxy, F, Cl, Br, I, —NH₂, —NH—C₁₋₅-alkyl, —N(C₁₋₅-alkyl)₂, —C(═O)—NH₂, —C(═O)—NH—C₁₋₅-alkyl, —C(═O)—N(C₁₋₅-alkyl)₂, —NO₂, CN, —CF₃, —CHF₂, —CH₂F, C₁₋₅ alkyl and C₁₋₅ alkoxy.

If residues R¹ and R² and/or R⁴ and R⁵ together with the nitrogen atom joining them together as a ring member form a cycloaliphatic residue with at least one further heteroatom, the heteroatoms, unless otherwise stated, may preferably be selected from the group consisting of oxygen, nitrogen and sulfur. The cyclic residues may preferably comprise 1 or 2 further heteroatoms as ring members.

If residues R¹ and R² and/or R⁴ and R⁵ together with the nitrogen atom joining them together as a ring member form a cycloaliphatic residue optionally comprising at least one further heteroatom as a ring member, which cycloaliphatic residue may be mono- or polysubstituted, said residue may preferably be selected from the group consisting of imidazolidinyl, aziridinyl, azetidinyl, pyrrolidinyl, piperidinyl, azepanyl, azocanyl, piperazinyl, morpholinyl and thiomorpholinyl.

If one or both of the above-stated residues R⁴ and R⁵ denote(s) a saturated or unsaturated cycloaliphatic residue optionally comprising at least one further heteroatom, for example 1 or 2 heteroatoms, as a ring member or comprise(s) such a residue which is identically or differently mono- or polysubstituted, for example mono-, di-, tri-, tetra- or pentasubstituted, the corresponding substituents may mutually independently preferably be selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH-C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

The particular furanyl, thiophenyl, pyridinyl, phenyl or benzyl substituent may itself be unsubstituted or mono- or polysubstituted, optionally mono-, di-, tri-, tetra- or pentasubstituted, wherein the substituents thereof may mutually independently preferably be selected from the group consisting of hydroxy, F, Cl, Br, I, —NH₂, —NH-C₁₋₅-alkyl, —N(C₁₋₅-alkyl)₂, —C(═O)—NH₂, —C(═O)—NH—C₁₋₅-alkyl, —C(═O)—N(C₁₋₅-alkyl)₂, —NO₂, —CN, —CF₃, —CHF₂, —CH₂F, C₁₋₅ alkyl and C₁₋₅ alkoxy. Unless otherwise stated, the heteroatoms may preferably be selected from the group consisting of oxygen, nitrogen and sulfur.

Suitable cycloaliphatic residues, which may be mono- or polysubstituted, for example mono-, di-, tri-, tetra- or pentasubstituted, and may be mentioned by way of example, include cyclopropyl, cyclobutyl, cyclopentyl, cyclohexyl, cyclohexyl, cyclooctyl, cyclononyl, cyclopentenyl, cyclohexenyl, cycloheptenyl or cyclooctenyl.

Suitable cycloaliphatic residues comprising one or more heteroatoms as a ring member, which cycloaliphatic residues may be mono- or polysubstituted, for example mono-, di-, tri-, tetra- or pentasubstituted, and may be mentioned by way of example include imidazolidinyl, aziridinyl, azetidinyl, pyrrolidinyl, piperidinyl, azepanyl, azocanyl, piperazinyl, tetrahydrofuranyl (tetrahydrofuryl), morpholinyl and thiomorpholinyl.

If one or both of the above-stated residues R⁴ and R⁵ denote(s) a mono- or polysubstituted, for example mono-, di-, tri-, tetra- or pentasubstituted aryl residue denote or comprise(s) such a residue, the corresponding substituents may mutually independently preferably be selected from the group consisting of C₁₋₅ alkyl, —C(═O)—O—C₁₋₅-alkyl, —O—C₁₋₅-alkyl, Cl, F, Br, I, —C(═O)—NH₂, —C(═O)—NH—C₁₋₅-alkyl, —C(═O)—N(C₁₋₅-alkyl)₂, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl. The particular furanyl, thiophenyl, pyridinyl, phenyl or benzyl substituent may itself be unsubstituted or mono- or polysubstituted, optionally mono-, di-, tri-, tetra- or pentasubstituted, wherein the substituents thereof may mutually independently preferably be selected from the group consisting of hydroxy, F, Cl, Br, I, —NH₂, —NH—C₁₋₅-alkyl, —N(C₁₋₅-alkyl)₂, —C(═O)—NH₂, —C(═O)—NH—C₁₋₅-alkyl, —C(═O)—N(C₁₋₅-alkyl)₂, —NO₂, —CN, —CF₃, —CHF₂, —CH₂F, C₁₋₅ alkyl and C₁₋₅ alkoxy.

Suitable aryl residues, which may be mono- or polysubstituted and which may be mentioned by way of example are phenyl, 1-naphthyl and 2-naphthyl.

The above-stated C₁₋₅ alkoxy and C₁₋₅ alkyl residues may in each case be linear or branched. The C₁₋₅ alkyl residues comprise the residues methyl, ethyl, n-propyl, iso-propyl, n-butyl, iso-butyl, sec-butyl, tert-butyl, n-pentyl, iso-pentyl and neopentyl, the C₁₋₅ alkoxy residues comprise the residues methoxy, ethoxy, n-propoxy, iso-propoxy, n-butoxy, iso-butoxy, sec-butoxy, tert-butoxy, n-pentoxy, iso-pentoxy and neopentoxy.

Very particularly preferred substituted 2,5-diaminomethyl-1H-pyrroles according to the invention are those selected from the group consisting of: diethyl-(1-methyl-5-piperidin-1-ylmethyl-1H-pyrrol-2-ylmethyl)-amine,
1-(1-methyl-5-piperidin-1-ylmethyl-1H-pyrrol-2-ylmethyl)-4-methylpiperidine,
4-[1-methyl-5-(4-phenyl-piperidin-1-ylmethyl)-1H-pyrrol-2-ylmethyl]-morpholine,
1-(1-methyl-5-piperidin-1-ylmethyl-1H-pyrrol-2-ylmethyl)-4-phenyl-piperazine,
1-[5-(4-benzyl-piperidin-1-ylmethyl)-1-methyl-1H-pyrrol-2-ylmethyl]-azepane,
benzyl-[5-(4-benzyl-piperidin-1-ylmethyl)-1-methyl-1H-pyrrol-2-ylmethyl]-methylamine,
4-[5-(4-benzyl-piperidin-1-ylmethyl)-1-methyl-1H-pyrrol-2-ylmethyl]-morpholine,
(5-azepan1-ylmethyl-1-methyl-1H-pyrrol-2-ylmethyl)-benzyl-methyl-amine,
benzyl-methyl-[1-methyl-5-(4-phenyl-piperidin-1-ylmethyl)-1H-pyrrol-2-ylmethyl]-amine and
benzyl-methyl-[1-methyl-5-(4-phenyl-piperidin1-ylmethyl)-1H-pyrrol-2-ylmethyl]-amine,
   in each case optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts or in each case in the form of corresponding solvates.

The present invention also provides a process for the production of substituted 2,5-diaminomethyl-1H-pyrroles according to the invention of formula I, in accordance with which a substituted aminomethyl-1H-pyrrole of formula II, 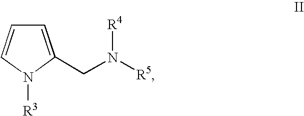
in which residues R³, R⁴ and R⁵ have the above-stated meaning, are reacted using conventional methods known to the person skilled in the art, preferably in a suitable reaction medium, such as for example CH₂Cl₂, CH₃CN, dimethylformamide (DMF) or mixtures of at least two of these solvents, at room temperature (approx. 20-25° C.) with an iminium salt of formula III, 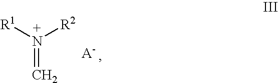
in which R¹ to R² have the above-stated meaning and A⁻ denotes a suitable anion, preferably Cl⁻, AlCl₄⁻, Br⁻, I⁻ or CF₃—SO₃⁻ (triflate anion), to yield a substituted 2,5-dimethylamino-1H-pyrrole according to the invention of formula I and this latter compound is optionally purified using conventional methods known to the person skilled in the art, preferably by extraction, and optionally isolated.

The compounds of formula II may be produced using conventional methods known to the person skilled in the art, for example from commercially obtainable reagents of formula IV, 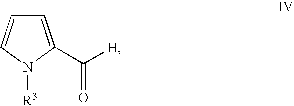
as for example described in A. F. Abdel-Magid et al., Journal of Organic Chemistry, 1996, 61, pages 3849-3862, the disclosure of which is incorporated herein by reference.

The iminium salts of formula III may likewise be obtained using conventional methods known to the person skilled in the art, for example from the corresponding aminals of the following formula V: 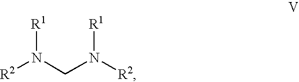
in which residues R¹ and R² have the above-stated meaning, as for example described in H. Heaney, Tetrahedron 1997, 53, pages 2941-2958 and H. Heaney, Tetrahedron Lett. 1988, 29, pages 2377-2380. The disclosures of these literature documents are hereby incorporated by reference.

The aminals of the general formula V may also be produced using methods known from the literature, as for example described in H. Heaney, Tetrahedron 1997, 53, pages 2941-2958 and H. Heaney, Tetrahedron Lett. 1988, 29, pages 2377-2380. The disclosures of these literature documents are also hereby incorporated by reference.

The substituted 2,5-diaminomethyl-1H-pyrroles of formula I and corresponding stereoisomers may be isolated not only in the form of the free bases or free acids thereof, but also in the form of corresponding salts, in particular physiologically acceptable salts.

The free bases of the particular substituted 2,5-diaminomethyl-1H-pyrroles according to the invention of formula I and corresponding stereoisomers may, for example, be converted into the corresponding salts, preferably physiologically acceptable salts, by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid.

The free bases of the particular substituted 2,5-diaminomethyl-1H-pyrroles according to the invention of formula I and corresponding stereoisomers may preferably be converted into the corresponding hydrochloride salts by combining the compounds of formula I or corresponding stereoisomers as free bases dissolved in a suitable organic solvent, such as for example butan-2-one (methyl ethyl ketone), with trimethylsilyl chloride (TMSCl).

The free bases of the particular substituted 2,5-diaminomethyl-1H-pyrroles of formula I and corresponding stereoisomers may likewise be converted into the corresponding physiologically acceptable salts with the free acid or a salt of a sugar substitute, such as for example saccharin, cyclamate or acesulfame.

The free acids of the substituted 2,5-diaminomethyl-1H-pyrroles of formula I and corresponding stereoisomers may accordingly be converted into the corresponding physiologically acceptable salts by reaction with a suitable base.

The substituted 2,5-diaminomethyl-1H-pyrroles according to the invention of formula I and corresponding stereoisomers may optionally, like the corresponding acids, the corresponding bases or salts of these compounds, also be obtained in the form of the solvates thereof, preferably the hydrates thereof, by conventional methods known to the person skilled in the art.

If the substituted 2,5-diaminomethyl-1H-pyrroles of formula I are obtained after the production thereof in the form of the racemates thereof or other mixtures of the various enantiomers and/or diastereomers thereof, these may be separated and optionally isolated by conventional methods known to the person skilled in the art. Examples which may be mentioned are chromatographic separation methods, in particular liquid chromatography methods at standard pressure or at elevated pressure, preferably MPLC and HPLC methods, and fractional crystallisation methods. Individual enantiomers, e.g. diastereomeric salts formed by means of HPLC on a chiral stationary phase or by means of crystallisation with chiral acids, such as (+)-tartaric acid, (−)-tartaric acid or (+)-10-camphorsulfonic acid, may here in particular be separated from one another.

The substituted 2,5-diaminomethyl-1H-pyrroles according to the invention of formula I including the above-excepted compounds and corresponding stereoisomers as well as in each case the corresponding acids, bases, salts and solvates are toxicologically safe and are therefore suitable as pharmaceutical active ingredients in pharmaceutical preparations.

The present invention accordingly further provides pharmaceutical preparations containing at least one substituted 2,5-diaminomethyl-1H-pyrrole according to the invention of formula I including the above-excepted compounds, optionally in the form of the racemate thereof, the pure stereoisomers thereof, in particular enantiomers or diastereomers, or in the form of mixtures of the stereoisomers, in particular the enantiomers and/or diastereomers, in any desired mixing ratio, or in each case in the form of a corresponding salt, in particular of the physiologically acceptable salt, particularly preferably of the hydrochloride salt, or a corresponding solvate, in particular of the hydrate, and optionally one or more physiologically acceptable auxiliary substances.

These pharmaceutical preparations according to the invention are in particular suitable for opioid receptor regulation, preferably for μ opioid receptor regulation, for regulating noradrenalin (NA) uptake, preferably for inhibiting noradrenalin (NA) uptake, and for regulating 5-hydroxytryptamine (5-HT) uptake, preferably for inhibiting 5-hydroxytryptamine (5-HT) uptake.

The pharmaceutical preparations according to the invention are likewise preferably suitable for the prevention and/or treatment of disorders or diseases, which are at least partially mediated by opioid receptors, in particular by μ opioid receptors, and/or noradrenalin (NA) receptors and/or 5-hydroxytryptamine (5-HT) receptors.

The pharmaceutical preparations according to the invention are likewise preferably suitable for the treatment of pain, preferably selected from the group consisting of chronic pain and/or acute pain and/or neuropathic pain, for the prevention and/or treatment of withdrawal symptoms, memory disorders, neurodegenerative diseases, preferably selected from the group consisting of Parkinson's disease, Huntington's chorea, Alzheimer's disease and multiple sclerosis, epilepsy, cardiovascular disorders, water retention conditions, intestinal motility (diarrhoea), urinary incontinence, anorexia, tinnitus, pruritus, depression, sexual dysfunction, preferably erectile dysfunction or airways diseases, disorders of food intake, preferably selected from the group consisting of obesity, bulimia, anorexia, cachexia and type II diabetes (non-insulin-dependent diabetes), or for anxiolysis, for diuresis, for suppressing the urinary reflex, for reducing the addictive potential of opioids, preferably morphine, for modulating locomotor activity, for influencing the cardiovascular system, preferably for vasodilating the arteries, or for regulating the electrolyte balance.

The pharmaceutical preparations according to the invention are particularly preferably suitable for the treatment of pain, preferably selected from the group consisting of chronic pain, acute pain and neuropathic pain.

The present invention also provides the use one or more substituted 2,5-diaminomethyl-1H-pyrroles of formula I including the above-excepted compounds, optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts, in particular the physiologically acceptable salts, particularly preferably the hydrochlorides, or in each case in the form of corresponding solvates, in particular the hydrates, for the production of a pharmaceutical preparation for opioid receptor regulation, preferably for μ opioid receptor regulation, for regulating noradrenalin (NA) uptake, preferably for inhibiting noradrenalin (NA) uptake or for regulating hydroxytryptamine (5-HT) uptake, preferably for inhibiting 5-hydroxytryptamine (5-HT) uptake.

The present invention also relates to the use of one or more substituted 2,5-diaminomethyl-1H-pyrroles corresponding to formula I, including the above-excepted compounds, optionally in the form of the pure stereoisomers thereof, in particular enantiomers or diastereomers, the racemates thereof or in the form of mixtures of the stereoisomers, in particular the enantiomers and/or diastereomers, in any desired mixing ratio, or in each case in the form of corresponding salts, in particular the physiologically acceptable salts, particularly preferably the hydrochlorides, or in each case in the form of corresponding solvates, in particular the hydrates, for the production of a pharmaceutical preparation for the treatment of pain, preferably selected from the group consisting of chronic pain, pain and neuropathic pain, for the prevention and/or treatment of withdrawal symptoms, memory disorders, neurodegenerative diseases, preferably selected from the group consisting of Parkinson's disease, Huntington's chorea, Alzheimer's disease and/or multiple sclerosis, epilepsy, cardiovascular disorders, water retention conditions, intestinal motility (diarrhoea), urinary incontinence, anorexia, tinnitus, pruritus, depression, sexual dysfunction, preferably erectile dysfunction, or airways diseases, disorders of food intake, preferably selected from the group consisting of obesity, bulimia, anorexia, cachexia and type II diabetes (non-insulin-dependent diabetes), or for anxiolysis, for diuresis, for suppressing the urinary reflex, for reducing the addictive potential of opioids, preferably morphine, for modulating locomotor activity, for influencing the cardiovascular system, preferably for vasodilating the arteries, or for regulating the electrolyte balance.

The pharmaceutical preparations according to the invention may assume the form of liquid, semisolid or solid dosage forms, for example in the form of solutions for injection, drops, succi, syrups, sprays, suspensions, tablets, patches, capsules, dressings, suppositories, ointments, creams, lotions, gels, emulsions, aerosols or in multiparticulate form, for example in the form of pellets or granules, optionally press-moulded into tablets, packaged in capsules or suspended in a liquid, and also be administered as such.

In addition to one or more substituted 2,5-diaminomethyl-1H-pyrroles of formula I including the above-excepted compounds, optionally in the form of the racemates thereof, the pure stereoisomers thereof, in particular enantiomers or diastereomers, or in the form of mixtures of the stereoisomers, in particular the enantiomers or diastereomers, in any desired mixing ratio, or in each case in the form of a corresponding salt, in particular a physiologically acceptable salt, or in the form of a corresponding solvate, in particular of the hydrate, the pharmaceutical preparations according to the invention conventionally contain further physiologically acceptable pharmaceutical auxiliary substances, which may preferably be selected from the group consisting of matrix materials, fillers, solvents, diluents, surface-active substances, dyes, preservatives, disintegrants, slip agents, lubricants, aromas and binders.

Selection of the physiologically acceptable auxiliary substances and the quantities thereof which are to be used depends upon whether the pharmaceutical preparation is to be administered orally, subcutaneously, parenterally, intravenously, intraperitoneally, intradermally, intramuscularly, intranasally, buccally, rectally or topically, for example onto infections of the skin, mucous membranes and eyes.

Preparations in the form of tablets, coated tablets, capsules, granules, pellets, drops, succi and syrups are preferred for oral administration, while solutions, suspensions, readily reconstitutible dried preparations and sprays are preferred for parenteral, topical and inhalatory administration.

Substituted 2,5-diaminomethyl-1H-pyrroles of formula I including the above-excepted compounds, optionally in the form of the racemates thereof, the pure stereoisomers thereof, in particular enantiomers or diastereomers, or in the form of mixtures of the stereoisomers, in particular the enantiomers or diastereomers, in any desired mixing ratio, or in each case in the form of a corresponding salt, in particular a physiologically acceptable salt, or in the form of a corresponding solvate, in particular the hydrate, in a depot in dissolved form or in a dressing, optionally with the addition of skin penetration promoters, are suitable percutaneous administration preparations.

Orally or percutaneously administrable formulations may also release the particular substituted 2,5-diaminomethyl-1H-pyrroles of formula I including the above-excepted compounds, optionally in the form of the racemates thereof, the pure stereoisomers thereof, in particular the enantiomers or diastereomers, or in the form of mixtures of the stereoisomers, in particular of the enantiomers or diastereomers, in any desired mixing ratio, or in each case in the form of a corresponding salt, in particular of a physiologically acceptable salt, or in the form of a corresponding solvate, in particular the hydrate, in delayed manner

Production of the pharmaceutical preparations according to the invention proceeds with the assistance of conventional means, devices, methods and processes known to the person skilled in the art, such as are described for example in A. R. Gennaro (ed.), “Remington's Pharmaceutical Sciences”, 17th edition, Mack Publishing Company, Easton, Pa. (1985), in particular in part 8, chapters 76 to 93. The corresponding literature description is hereby introduced as a reference and is deemed to be part of the disclosure.

The quantity to be administered to the patient of the particular substituted 2,5-dimethylamino-1H-pyrrole of formula I including the above-excepted compounds, optionally in the form of the racemate thereof, the pure stereoisomer thereof, in particular enantiomer or diastereomer, or in the form of mixtures of the stereoisomers, in particular the enantiomers or diastereomers, in any desired mixing ratio or in each case in the form of a corresponding salt, in particular a physiologically acceptable salt, or in each case of a corresponding solvate thereof, in particular the hydrate, may vary and is for example dependent on the weight or age of the patient and on the mode of administration, the indication and the severity of the complaint. Conventionally, 0.005 to 500 mg/kg, preferably 0.05 to 50 mg/kg of patient body weight of at least one substituted 2,5-dimethylamino-1H-pyrrole of formula I including the above-excepted compounds, optionally in the form of the racemate thereof, the pure stereoisomers thereof, in particular enantiomers or diastereomers, or in the form of mixtures of the stereoisomers, in particular the enantiomers or diastereomers, in any desired mixing ratio, or in each case in the form of a corresponding salt, in particular a physiologically acceptable salt, or in the form of a corresponding solvate, in particular the hydrate, are administered.

Pharmacological Methods

a) Method for determining affinity for human μ opioid receptor

Receptor affinity for the human μ opioid receptor is determined in a homogeneous batch in microtitre plates. To this end, dilution series of the particular substituted 2,5-diaminomethyl-1H-pyrrole of formula I to be tested were incubated at room temperature for 90 minutes in a total volume of 250 μl with a receptor membrane preparation (15-40 μg of protein per 250 μl of incubation batch) of CHO-K1 cells, which express the human μ opioid receptor (μ opiate receptor) (RB-HOM receptor membrane preparation from NEN, Zaventem, Belgium) in the presence of 1 nmol/l of the radioactive ligand [³H]-naloxone (NET719, from NEN, Zaventem, Belgium) and of 1 mg of WGA-SPA beads (wheat germ agglutinin SPA beads from Amersham/Pharmacia, Freiburg, Germany). The incubation buffer used is 50 mmol/l tris-HCl supplemented with 0.05 wt. % of sodium azide and with 0.06 wt. % of bovine serum albumin. 25 μmol/l of naloxone were additionally added to determine nonspecific binding. Once the ninety minute incubation time had elapsed, the microtitre plates were centrifuged off for 20 minutes at 1000 g and the radioactivity measured in a β-Counter (Microbeta-Trilux, from PerkinElmer Wallac, Freiburg, Germany). The percentage displacement of the radioactive ligand from its binding to the human μ opiate receptor is determined at a concentration of the compounds to be tested of 1 μmol/l and stated as percentage inhibition of specific binding. On the basis of the percentage displacement by different concentrations of the compounds to be tested of formula I, IC₅₀ inhibition concentrations which bring about 50% displacement of the radioactive ligand were calculated. Kᵢ values for the test substances may be obtained by conversion using the Cheng-Prusoff equation.

b) Method For Determining Noradrenalin and 5-HT Uptake Inhibition

Synaptosomes from rat brain regions are freshly isolated for in vitro studies, as described in the publication “The isolation of nerve endings from brain” by E. G. Gray and V. P. Whittaker, J. Anatomy 96, pages 79-88, 1962. The corresponding literature description is hereby introduced as a reference and is deemed to be part of the disclosure.

The tissue (hypothalamus for the determination of noradrenalin uptake inhibition and medulla and pons for the determination of 5-HT uptake inhibition) is homogenised in ice-cooled 0.32 M sucrose (100 mg of tissue/1 ml) in a glass homogeniser with Teflon pestle using five complete up and down strokes at 840 revolutions/minute.

The homogenate is centrifuged at 4° C. for 10 minutes at 1000 g. After subsequent centrifugation at 17000 g for 55 minutes, the synaptosomes (P₂ fraction) are obtained, which are resuspended in 0.32 M glucose (0.5 ml/100 mg of original weight).

The particular uptake is measured in a 96-well microtitre plate. The volume is 250 μl and the incubation proceeds at room temperature (approx. 20-25° C.) under an O₂ atmosphere.

The incubation time is 7.5 minutes for [³H]—NA and 5 minutes for [³H]-5-HT. The 96 samples are then filtered through a Unifilter GF/B® microtitre plate (Packard) and washed with 200 ml of incubated buffer using a “Brabdel MPXRI-96T Cell-Harvester”. The Unifilter GF/B plate is dried for 1 hour at 55° C. The plate is then sealed with a Back seal® (Packard) and 35 μl of scintillation fluid are added per well (Ultima Gold®, Packard). After sealing with a top seal® (Packard) and establishing an equilibrium (around 5 hours), radioactivity is determined in a “Trilux 1450 Microbeta” (Wallac).

The quantity of protein used in the above determination corresponds to the values known from the literature, as for example described in “Protein measurement with the folin phenol reagent”, Lowry et al., J. Biol. Chem., 193, 265-275, 1951. A detailed description of the method may additionally be found in the literature, for example in M.Ch. Frink, H.-H. Hennies, W. Engelberger, M. Haurand and B. Wilffert (1996) Arzneim.-Forsch./Drug Res. 46 (III), 11, 1029-1036. The disclosures of these two articles are hereby incorporated by reference.

The following characteristics were determined for the NA or 5-HT transporter:
NA uptake: Km=0.32±0.11 μM
5HT uptake: Km=0.084±0.011 μM

c) Investigation of Analgesic Efficacy By The Writhing Test

Investigation of the compounds according to the invention of formula I for analgesic efficacy is performed by phenylquinone-induced writhing in the mouse, modified after I. C. Hendershot and J. Forsaith (1959) J. Pharmacol. Exp. There. 125, 237-240. The corresponding literature description is hereby introduced as a reference and is deemed to be part of the disclosure.

Male NMRI mice weighing from 25 to 30 g were used for this purpose. Groups of 10 animals per compound dose receive, 10 minutes after intravenous administration of the compounds to be tested, 0.3 ml/mouse of a 0.02% aqueous solution of phenylquinone (phenylbenzoquinone, Sigma, Deisenhofen, Germany; solution prepared with addition of 5% of ethanol and stored in a water bath at 45° C.) administered intraperitoneally. The animals are placed individually in observation cages. A push button counter is used to record the number of pain-induced stretching movements (writhing reactions=straightening of the torso with stretching of the rear extremities) for 5-20 minutes after phenylquinone administration. The control was provided by animals which had received only physiological common salt solution. All the compounds were tested at the standard dosage of 10 mg/kg.

The invention is explained in further detail below with reference to illustrative examples, which are given merely by way of example and are not intended to restrict the general concept and scope of the invention.

### Examples

The NMR spectra were measured on a Bruker DPX 300 instrument for the 300 MHz spectra and on a Bruker DRX 600 instrument for the 600 MHz spectra. The particular chemicals and solvents were purchased from conventional commercial sources.

A) General synthesis method for the production of compounds of formula II:

The particular pyrrole carbaldehyde (90 mmol) of formula IV was dissolved in 600 ml of tetrahydrofuran and combined in succession with the corresponding amine of the general formula HNR⁴R⁵ (90 mmol) and sodium borohydride triacetate (NaBH(OAc)₃ (126 mmol)). After 20 hours' stirring at room temperature (approx. 20-25° C.), the reaction mixture was evaporated in a rotary evaporator. The resultant residue was redissolved in 500 ml of water and 200 ml of diethyl ether and adjusted to pH 4-5 with glacial acetic acid (30 ml). Extraction was performed three times with 100 ml portions of diethyl ether. The aqueous phase was then adjusted to pH 8 with saturated sodium hydrogencarbonate solution and extracted five times with 100 ml portions of diethyl ether. The organic phase was dried with magnesium sulfate and evaporated in a rotary evaporator.

The compounds of formula 11 produced by the above general synthesis method are listed in the following Table 1:

TABLE 1
Compounds of formula II

| Compounds | R⁴ | R⁵ | R³ | Yield [%] |
|---|---|---|---|---|
| II-1 | ]—(CH₂)₂—O—(CH₂)₂—[ | | CH₃ | 87 |
| II-2 | ]—(CH₂)₅—[ | | CH₃ | 70 |
| II-3 | CH₂CH₃ | CH₂CH₃ | CH₃ | 55 |
| II-4 | CH₂phenyl | CH₃ | CH₃ | 33 |

| | | | |
|---|---|---|---|
| II-5 | ]—(CH₂)₆—[ | CH₃ | 61 |

The structures of compounds II-1 to II-5, respectively, were determined by ¹H-NMR spectroscopy. The NMR data for compounds II-1 to II-3 correspond to the data known from the literature, as stated in W. Herz et al. Journal of the American Chemical Society 1951, 73, pages 4921-4923. The chemical shifts of selected compounds are shown below.

II-4 Benzyl-methyl-(1-methyl-1H-pyrrol-2-ylmethyl)-amine

δ (DMSO, 300 MHz)=2.11 (s, 3H, N(CH₃)CH₂Ph); 3.41-3.44 (m, 2H, N(CH₃)CH₂Ph); 3.45-3.48 (m, 2H, —CH₂—N—); 5.99-6.03 (m, 2H, N(CH₃) -]—CHCHCHC—[); 6.53-6.58 (m, 1H, N(CH₃)-]—CHCHCHC—[); 7.17-7.32 (m, 5H, Ph).

II-5 1-(1-Methyl-1H-pyrrol-2-ylmethyl)-azepane δ (DMSO, 300 MHz)=1.52-1.65 (m, 8H, N(CH₂)₂(CH₂)₂(CH₂)₂); 2.51-2.60 (m, 4H, N(CH₂)₂(CH₂)₂(CH₂)₂); 3.49-3.52 (m, 2H, —CH₂—N—); 3.62 (s, 3H, N—CH₃); 5.91-5.98 (m, 1H, N(CH₃)-]—CHCHCHC—[); 5.99-6.04 (m, 1H, N(CH₃)-]—CHCHCHC—[); 6.53-6.61 (m, 1H, N(CH₃)-]—CHCHCHC—[).

B) General procedure for the synthesis of the compounds according to the invention of formula I

Equimolar quantities of the particular iminium salt of formula III and of the particular aminomethyl-1H-pyrrole of formula II were initially introduced into dichloromethane in a flask and stirred for 16 hours at room temperature. Working up was performed by initially acidifying with HCl solution and extracting the non-basic impurities with diethyl ether or tert-butyl methyl ether. The aqueous phase was then neutralised with Na₂CO₃ solution and the product was extracted with diethyl ether or tert-butyl methyl ether. After drying the organic phase over magnesium sulfate, the product was obtained, which, for the purpose of further purification, was converted into the dihydrochloride with the assistance of ethanolic HCl solution, the dihydrochloride being washed repeatedly with cold ethanol.

The following example of the compounds according to the invention of formula I was produced in this manner:

### Example 1

Diethyl-(1-methyl-5-piperidin-1-ylmethyl-1H-pyrrol-2-ylmethyl)-amine dihydrochloride

δ (DMSO, 600 MHz)=1.23-1.31 (m, 6H, N(CH₂CH₃)₂, 1.33-1.41 (m, 1H, N(CH₂CH₂)₂CH₂); 1.64-1.72 (m, 1H, N(CH₂CH₂)₂CH₂); 1.73-1.86 (m, 4H, N(CH₂CH₂)₂CH₂); 2.86-2.94 (m, 2H, N(CH₂CH₂)₂CH₂); 3.02-3.17 (m, 4H, N(CH₂CH₃)₂, N(CH₂CH₃)₂); 3.26-3.41 (m, 2H, N(CH₂CH₂)₂CH₂); 3.78 (s, 3H, NCH₃); 4.24-4.35 (m, 4H, CH₂N(CH₂CH₂)₂CH₂, CH₂N(CH₂CH₃)₂); 6.36-6.46 (m, 2H, N(CH₃)]—CCHCHC—[); 10.19 (s, 1H, HCl); 10.26 (s, 1H, HCl).

General procedure for the automated synthesis of the compounds according to the invention of formula I. Synthesis proceeded in an automated synthesiser from Zymark.

Batch quantities: • 200 μmol of pyrrole of formula II
• 200 μmol of iminium salt of formula III

Pipetting volume: • 1 ml of pyrrole solution
• 1 ml of iminium salt solution

Stock solutions: • 0.2 M pyrrole solution in MeCN (solution I)
0.2 M iminium salt solution in DMF (solution II)
   Synthesis Procedure

200 μmol of iminium salt of formula III (solution II, 1 ml) were initially introduced at 20° C. into a dry threaded glass vial with a septum cap and combined with 200 μmol of pyrrole derivative (solution I, 1 ml). The reaction solution was stirred for 16 hours at 18° C. 2 ml of HCl solution (0.1 M) and 2 ml of dichloromethane were then added at 20° C. The reaction solution was intermixed for 30 minutes in the spin reactor. The magnetic stir bar was removed and the vessel rinsed out with 2 ml of dichloromethane.

Synthesis Work-up

The organic phase was removed and discarded 4 ml of dichloromethane were added in a vortexer and then intermixed for a further 10 minutes in the spin reactor. After centrifugation, the organic phase was again removed and discarded, the aqueous phase was combined once more with 4 ml of dichloromethane and adjusted to pH 8-9 with 0.7 ml of 7.5% strength NaHCO₃ solution. The solution was vigorously intermixed in the spin reactor, and, after centrifugation, the organic phase was separated and collected. The aqueous phase was extracted once more in a similar manner with 4 ml of dichloromethane. The combined organic phases were then dried over an MgSO₄ cartridge and evaporated under reduced pressure.

The following examples of the compounds according to the invention corresponding to formula I were produced in like manner:

### Example 2

1-(1-Methyl-5-piperidin-1-ylmethyl-1H-pyrrol-2-ylmethyl)-4-methylpiperidine

MS: (ESI) m/z=290 [M⁺+1]; 205; 191; 108.

### Example 3

4-[1-Methyl-5-(4-phenyl-piperidin-1-ylmethyl)-1H-pyrrol-2-ylmethyl]-morpholine

MS: (ESI) m/z=355 [M⁺+1]; 193; 108.

### Example 4

1-(1-Methyl-5-piperidin-1-ylmethyl-1H-pyrrol-2-ylmethyl)-4-phenyl-piperazine

MS: (ESI) m/z=353 [M⁺+1]; 268; 190; 108.

### Example 5

1-[5-(4-Benzyl-piperidin-1-ylmethyl)-1-methyl-1H-pyrrol-2-ylmethyl]-azepane

MS: (ESI) m/z=380 [M⁺+1]; 281; 205; 108.

### Example 6

Benzyl-[5-(4-benzyl-piperidin-1-ylmethyl)-1-methyl-1H-pyrrol-2-ylmethyl]-methyl-amine

MS: (ESI) m/z=402 [M⁺+1]; 281; 227

### Example 7

4-[5-(4-Benzyl-piperidin-1-ylmethyl)-1-methyl-1H-pyrrol-2-ylmethyl]-morpholine

MS: (ESI) m/z=368 [M⁺+1]; 281; 193; 108.

### Example 8

(5-Azepan-1-ylmethyl-1-methyl-1H-pyrrol-2-ylmethyl)-benzyl-methyl-amine

MS: (ESI) m/z=326 [M⁺+1]; 227; 205; 108.

### Example 9

Benzyl-methyl-[1-methyl-5-(2-methyl-piperidin-1-ylmethyl)-1H-pyrrol-2-ylmethyl]-amine

MS: (ESI) m/z=326 [M⁺+1]; 227; 205; 108.

### Example 10

Benzyl-methyl-[1-methyl-5-(4-phenyl-piperidin-1-ylmethyl)-1H-pyrrol-2-ylmethyl]-amine

MS: (ESI) m/z=326 [M⁺+1]; 227; 108.

Pharmacological Data

b) 5-HT uptake inhibition and noradrenalin (NA) reuptake inhibition

The 5-HT uptake inhibition and noradrenalin uptake inhibition of the 2,5-dimethylamino-1H-pyrroles according to the invention of formula I were determined as described above. The values for some selected compounds are shown in the following Table 2:

TABLE 2

| | | | | | | 5HT | NA |
|---|---|---|---|---|---|---|---|
| Compound | | | | | | uptake | uptake |
| according | | | | | | inhibition | inhibition |
| to Example | R¹ | R² | R⁴ | R⁵ | R³ | [2] | [3] |

| 1 | ]—(CH₂)₅-[ | C₂H₅ | C₂H₅ | CH₃ | 34 | |
|---|---|---|---|---|---|---|
| 2 | ]—(CH₂)₅-[-4-CH₃ | ]—(CH₂)₅—[ | | CH₃ | 72 | |
| 3 | ]—(CH₂)₄-[-4N- | ]—(CH₂)₂O(CH₂)₂—[ | | CH₃ | 59 | 71 |
| | phenyl | | | | | |
| 4 | ]—(CH₂)₄-[-4N- | ]—(CH₂)₅—[ | | CH₃ | 48 | 77 |
| | phenyl | | | | | |
| 5 | ]—(CH₂)₅-[-4- | ]—(CH₂)₆—[ | | CH₃ | 64 | 100 |
| | CH₂phenyl | | | | | |
| 6 | ]—(CH₂)₅-[-4- | CH₃ | CH₂phenyl | CH₃ | 61 | 100 |
| | CH₂phenyl | | | | | |
| 7 | ]—(CH₂)₅-[-4- | ]—(CH₂)₂O(CH₂)₂—[ | | CH₃ | 55 | 100 |
| | CH₂phenyl | | | | | |
| 8 | ]—(CH₂)₆-[ | CH₃ | CH₂phenyl | CH₃ | | 62 |
| 9 | ]—(CH₂)₅-[-2-CH₃ | CH₃ | CH₂phenyl | CH₃ | | 57 |
| 10 | ]—(CH₂)₅-[-4-phenyl | CH₃ | CH₂phenyl | CH₃ | | 53 |

[2] 5-HT uptake, rat, 10 μM, % inhibition,
[3] NA uptake, rat, 10 μM inhibition.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A substituted 2,5-diaminomethyl-1H-pyrrole compound corresponding to formula I: 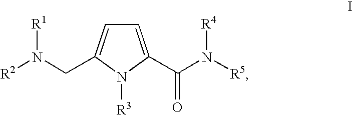
wherein
R¹ and R² together with the nitrogen atom to which they are attached form a saturated or unsaturated cycloaliphatic residue optionally comprising at least one further heteroatom as a ring member, which cycloaliphatic residue may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent optionally may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
R³ denotes a linear or branched, unsaturated or saturated, unsubstituted or at least monosubstituted aliphatic residue;
R⁴ denotes hydrogen, a linear or branched, unsubstituted or at least monosubstituted, unsaturated or saturated aliphatic residue, an unsaturated or saturated, unsubstituted or at least monosubstituted, cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched alkylene group;
R⁵ denotes a linear or branched, unsubstituted or at least monosubstituted, unsaturated or saturated aliphatic residue, an unsaturated or saturated, unsubstituted or at least monosubstituted cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched C₁₋₃ alkylene group, or
R⁴ and R⁵ together with the nitrogen atom to which they are attached form a saturated or unsaturated cycloaliphatic residue optionally comprising at least one further heteroatom as a ring member, which cycloaliphatic residue optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aliphatic residues may be substituted with substituents independently selected from the group consisting of halogen, hydroxy, —CN, —CF₃, —CHF₂, —CH₂F, C₁₋₆ alkoxy and optionally at least monosubstituted phenyl, the substituents of which may respectively be independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aryl residues may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂—NO₂, —CN, —CF₃, —CHF₂, —CH₂F, C₁₋₅ alkyl and C₁₋₅ alkoxy; and
wherein the above-stated cycloaliphatic residues may be substituted in the position of residues R⁴ and R⁵ with at least one substituent independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or substituted with at least one substituent independently selected from the group consisting of hydroxy, F, Cl, Br, I, —NH₂, —NH-C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
in the form of a pure stereoisomer thereof or a mixture of stereoisomers in any mxing ratio, or a salt or solvate thereof;
with the proviso that compounds are excluded in which:
residues R¹ and R² and residues R⁴ and R⁵, respectively, together with the nitrogen atoms to which they are attached form the same residue selected from the group consisting of pyrrolidinyl, morpholinyl and piperidinyl, and R³ denotes a methyl group, or
R¹ and R² together with the nitrogen atom to which they are attached form a piperidinyl residue, and R³, R⁴ and R⁵ each denote a methyl group.

2. A compound according to claim 1, wherein said compound is present in the form of a pure enantiomer or diastereomer.

3. A compound according to claim 1, wherein said compound is present in the form of a racemic mixture.

4. A compound according to claim 1, wherein R¹ and R² together with the nitrogen atom to which they are attached form a saturated or unsaturated 4-, 5-, 6-, 7-, 8- or 9-membered cycloaliphatic residue optionally comprising at least one further heteroatom as a ring member, which cycloaliphatic residue optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₅-alkyl, —N(C₁₋₅-alkyl)₂, —NO₂, —CN, —CF₃, —CHF₂, —CH₂F, C₁₋₅ alkyl and C₁₋₅ alkoxy.

5. A compound according to claim 4, wherein R¹ and R² together with the nitrogen atom to which they are attached form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which cycloaliphatic residue optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

6. A compound according to claim 4, wherein R¹ and R² together with the nitrogen atom to which they are attached form an imidazolidinyl, aziridinyl, azetidinyl, pyrrolidinyl, piperidinyl, azepanyl, azocanyl, piperazinyl, morpholinyl or thiomorpholinyl residue, which residue optionally may be identically or differently mono- or polysubstituted with at least one substituent selected from the group consisting of C₁₋₅ alkyl, —C(═O)—O—C₁₋₅-alkyl, O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl.

7. A compound according to claim 1, wherein R³ denotes a linear or branched, saturated or unsaturated, unsubstituted or at least monosubstituted aliphatic C₁₋₁₀ residue, wherein any substituents are independently be selected from the group consisting of halogen, hydroxy, —CN, —CF₃, —CHF₂, —CH₂F, C₁₋₆-alkoxy and optionally at least monosubstituted phenyl, the substituents of which are independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy.

8. A compound according to claim 7, wherein R³ denotes a linear or branched, unsubstituted or at least monosubstituted C₁₋₅ alkyl residue, wherein any substituents are independently selected from the group consisting of halogen, hydroxy, —CN, —CF₃, —CHF₂, —CH₂F, C₁₋₆ alkoxy and optionally at least monosubstituted phenyl, the substituents of which are independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy.

9. A compound according to claim 8, wherein R³ denotes an alkyl residue selected from the group consisting of methyl, ethyl, n-propyl, iso-propyl, n-butyl, iso-butyl, sec-butyl and tert-butyl.

10. A compound according to claim 1, wherein R⁴ denotes:
hydrogen, a linear or branched, saturated or unsaturated, unsubstituted or at least monosubstituted aliphatic C₁₋₁₀ residue, wherein any substituents are independently selected from the group consisting of halogen, hydroxy, —CN, —CF₃, —CHF₂, —CH₂F, C₁₋₆ alkoxy and optionally at least monosubstituted phenyl, the substituents of which are independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy, or
a saturated or unsaturated, unsubstituted or at least monosubstituted 3-, 4-, 5-, 6-, 7-, 8- or 9-membered cycloaliphatic residue optionally comprising at least one heteroatom as a ring member, and optionally attached via a linear or branched C₁₋₃ alkylene group, or
an unsubstituted or at least monosubstituted 5- to 14-membered aryl residue attached via a linear or branched C₁₋₃ alkylene group;
wherein the above-stated aryl residues may be substituted with at least one substituent independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂—NO₂, —CN, —CF₃, —CHF₂, —CH₂F, C₁₋₅ alkyl and C₁₋₅ alkoxy; and
wherein the above-stated cycloaliphatic residues may be substituted with at least one substituent independently selected from the group consisting of C₁₋₅ alkyl, —C(═O)—O—C₁₋₅ -alkyl, —O—C₁₋₅ -alkyl, Cl, F, Br, I, —C(═O)—C₁₋₅-alkyl, —NH₂, —NH—C₁₋₅-alkyl, —N(C₁₋₅ -alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or substituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy.

11. A compound according to claim 10, wherein R⁴ denotes hydrogen, a linear or branched C₁₋₅ alkyl residue, a saturated or unsaturated 4-, 5-, 6-, 7-, 8- or 9-membered cycloaliphatic residue, which may be attached via a linear or branched C₁₋₃ alkylene group, or an unsubstituted or at least monosubstituted phenyl residue attached via a linear or branched C₁₋₃ alkylene group.

12. A compound according to claim 11, wherein R⁴ denotes hydrogen, an alkyl residue selected from the group consisting of methyl, ethyl, propyl, iso-propyl, n-butyl, iso-butyl, sec-butyl and tert-butyl, or a benzyl residue.

13. A compound according to claim 1, wherein R⁵ denotes:
a linear or branched, saturated or unsaturated, unsubstituted or at least monosubstituted aliphatic C₁₋₁₀ residue, wherein any substituents are independently selected from the group consisting of halogen, hydroxy, —CN, —CF₃, —CHF₂, —CH₂F, C₁₋₆ alkoxy and optionally at least monosubstituted phenyl, the substituents of which are independently selected from the group consisting of hydroxy, F, Cl, Br, I, —NH₂, —NH—C₁₋₅-alkyl, —N(C₁₋₅ -alkyl)₂, —NO₂, —CN, —CF₃, —CHF₂, —CH₂F, C₁₋₅ alkyl, and C₁₋₅ alkoxy; or
a saturated or unsaturated, unsubstituted or at least monosubstituted 3-, 4-, 5-, 6-, 7-, 8- or 9-membered cycloaliphatic residue optionally comprising at least one heteroatom as a ring member, which optionally may be attached via a linear or branched C₁₋₃ alkylene group, or
an unsubstituted or at least monosubstituted 5- to 14-membered aryl residue attached via a linear or branched C₁₋₃ alkylene group;
wherein the above-stated aryl residues optionally may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂ —NO₂, —CN, —CF₃, —CHF₂, —CH₂F, C₁₋₅ alkyl und C₁₋₅ alkoxy; and
wherein the above-stated cycloaliphatic residues optionally may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself optionally be unsubstituted or substituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy.

14. A compound according to claim 13, wherein R⁵ denotes a linear or branched C₁₋₅ alkyl residue, a saturated or unsaturated 4-, 5-, 6-, 7-, 8- or 9-membered cycloaliphatic residue, which optionally may be attached via a linear or branched C₁₋₃ alkylene group, or an unsubstituted or at least monosubstituted phenyl residue attached via a linear or branched C₁₋₃ alkylene group.

15. A compound according to claim 14, wherein R⁵ denotes an alkyl residue selected from the group consisting of methyl, ethyl, propyl, iso-propyl, n-butyl, iso-butyl, sec-butyl and tert-butyl or a benzyl residue.

16. A compound according to claim 1, wherein R⁴ and R⁵ together with the nitrogen atom to which they are attached form a saturated or unsaturated 4-, 5-, 6-, 7-, 8 or 9-membered cycloaliphatic residue optionally comprising at least one further heteroatom as a ring member, which cycloaliphatic residue optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅ alkyl, —N(C₁₋₅-alkyl)₂, —NO₂, —CN, —CF₃, —CHF₂, —CH₂F, C₁₋₅ -alkyl and C₁₋₅- alkoxy.

17. A compound according to claim 16, wherein R⁴ and R⁵ together with the nitrogen atom to which they are attached form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which cycloaliphatic residue optionally may be identically or differently mono- or polysubstituted with at least one substituent selected from the group consisting of C₁₋₅-alkyl, —C(═O)—O—C₁₋₅-alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅ alkyl, —N(C₁₋₅-alkyl)₂, —NO₂, —CN, —CF₃, —CHF₂, —CH₂F, C₁₋₅ alkyl and C₁₋₅ alkoxy.

18. A compound according to claim 1, wherein R⁴ and R⁵ together with the nitrogen atom to which they are attached form an imidazolidinyl, aziridinyl, azetidinyl, pyrrolidinyl, piperidinyl, azepanyl, azocanyl, piperazinyl, morpholinyl or thiomorpholinyl residue, which optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅ alkyl, —N(C₁₋₅-alkyl)₂, —NO₂, —CN, —CF₃, —CHF₂, —CH₂F, C₁₋₅-alkyl and C₁₋₅-alkoxy.

19. A compound according to claim 1, wherein:
R¹ and R² together with the nitrogen atom to which they are attached form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which cycloaliphatic residue optionally may be identically or differently mono- or polysubstituted with at least one substituent selected from the group consisting of C₁₋₅ alkyl, —C(═O)—O—C₁₋₅ alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself optionally be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
R³ denotes a linear or branched, unsubstituted or at least monosubstituted C₁₋₅ alkyl residue, wherein any substituents are independently selected from the group consisting of halogen, hydroxy, —CN, —CF₃, —CHF₂, —CH₂F, C₁₋₆ alkoxy and optionally at least monosubstituted phenyl, the substituents of which are independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
R⁴ denotes hydrogen, a linear or branched C₁₋₅ alkyl residue, a saturated or unsaturated 4-, 5-, 6-, 7-, 8- or 9-membered cycloaliphatic residue, which optionally may be attached via a linear or branched C₁₋₃ alkylene group, or an unsubstituted or at least monosubstituted phenyl residue attached via a linear or branched C₁₋₃ alkylene group; and
R⁵ denotes a linear or branched C₁₋₅ alkyl residue, a saturated or unsaturated 4-, 5-, 6-, 7-, 8- or 9-membered cycloaliphatic residue, which optionally may be attached via a linear or branched C₁₋₃ alkylene group, or an unsubstituted or at least monosubstituted phenyl residue attached via a linear or branched C₁₋₃ alkylene group; or
R⁴ and R⁵ together with the nitrogen atom to which they are attached form a saturated or unsaturated 4-, 5-, 6-, 7-, 8- or 9-membered cycloaliphatic residue optionally comprising at least one further heteroatom selected from the group consisting of nitrogen, oxygen and sulfur as a ring member, which optionally may be identically or differently mono- or polysubstituted with at least one substituent selected from the group consisting of C₁₋₅ alkyl, —C(═O)—O—C₁₋₅-alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent optionally may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above stated aryl residues optionally may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent optionally may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂ —NO₂, —CN, —CF₃, —CHF₂, —CH₂F, C₁₋₅ alkyl and C₁₋₅ alkoxy.

20. A compound according to claim 1, wherein:
R¹ and R² together with the nitrogen atom to which they are attached form an imidazolidinyl, aziridinyl, azetidinyl, pyrrolidinyl, piperidinyl, azepanyl, azocanyl, piperazinyl, morpholinyl or thiomorpholinyl residue, which optionally may be substituted with at least one substituent selected from the group consisting of methyl, ethyl, n-propyl, iso-propyl, n-butyl, iso-butyl, sec-butyl, tert.-butyl, phenyl and benzyl;
R³ denotes an alkyl residue selected from the group consisting of methyl, ethyl, n-propyl, iso-propyl, n-butyl, iso-butyl, sec-butyl and tert-butyl;
R⁴ denotes hydrogen, an alkyl residue selected from the group consisting of methyl, ethyl, n-propyl, iso-propyl, n-butyl, iso-butyl, sec-butyl, tert-butyl, n-pentyl, iso-pentyl and neopentyl, or a benzyl residue; and
R⁵ denotes an alkyl residue selected from the group consisting of methyl, ethyl, n-propyl, iso-propyl, n-butyl, iso-butyl, sec-butyl, tert-butyl, n-pentyl, iso-pentyl and neopentyl, or a benzyl residue; or
R⁴ and R⁵ together with the nitrogen atom to which they are attached form an imidazolidinyl, aziridinyl, azetidinyl, pyrrolidinyl, piperidinyl, azepanyl, azocanyl, piperazinyl, morpholinyl or thiomorpholinyl residue, which optionally may be substituted with at least one substituent selected from the group consisting of methyl, ethyl, n-propyl, iso-propyl, n-butyl, iso-butyl, sec-butyl, tert-butyl, phenyl and benzyl.

21. A compound according to claim 1, selected from the group consisting of:
diethyl-(1-methyl-5-piperidin-1-ylmethyl-1H-pyrrol-2-ylmethyl)-amine;
1-(1-methyl-5-piperidin-1-ylmethyl-1H-pyrrol-2-yl methyl )-4-methyl piperidine;
4-[1-methyl-5-(4-phenyl-piperidin-1-ylmethyl)1H -pyrrol-2-ylmethyl]-morpholine;
1-(1-methyl-5-piperidin-1-ylmethyl-1H-pyrrol-2-ylmethyl)-4-phenyl-piperazine;
1-[5-(4-benzyl-piperidin-1-ylmethyl)-1-methyl-1H-pyrrol-2-ylmethyl]-azepane;
benzyl-[5-(4-benzyl-piperidin-1-ylmethyl)-1-methyl-1H-pyrrol-2-ylmethyl]-methylamine;
4-[5-(4-benzyl-piperidin-1-ylmethyl)-1-methyl-1H-pyrrol-2-yl methyl]-morpholine;
(5-azepan1-ylmethyl1-methyl-1H-pyrrol-2-ylmethyl)-benzyl-methyl-amine;
benzyl-methyl-[1-methyl-5-(4-phenyl-piperidin-1-ylmethyl)-1H-pyrrol-2-ylmethyl]-amine, and
benzyl-methyl-[1-methyl-5-(4-phenyl-piperidin-1-ylmethyl)-1H-pyrrol-2-ylmethyl]-amine;
in the form of a pure stereoisomer or a mixture of stereoisomers in any mixing ratio, or a salt or hydrate thereof.

22. A process for producing a 2,5-diaminomethyl-1H-pyrrole compound according to claim 1, said process comprising:
reacting a compound of compound of formula II: 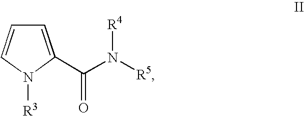
wherein R³, R⁴ and R⁵ have the meanings given in claim 1, with an iminium salt of formula III: 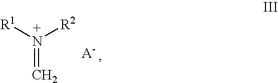
wherein R¹ and R² have the meanings given in claim 1, and A⁻ denotes an anion, to yield a 2,5-diaminomethyl-1H-pyrrole reaction product; and
optionally optionally isolating or purifying the reaction product.

23. A process according to claim 22, wherein A⁻ denotes an anion selected from the group consisting of Cl⁻, AlCl₄⁻, Br⁻, I⁻ and CF₃—SO₃⁻.

24. A pharmaceutical composition comprising a compound according to claim 1, and at least one pharmaceutically acceptable carrier or auxiliary substance.

25. A method of regulating a physiological process selected from the group consisting of 5-hydroxytryptamine uptake, noradrenalin uptake, and opioid receptor activity in a patient in need thereof, said method comprising administering to said patient a pharmaceutically effective amount of a substituted 2,5-diaminomethyl-1H-pyrrole compound corresponding to formula I: 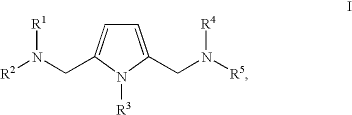
wherein
R¹ and R² together with the nitrogen atom to which they are attached form a saturated or unsaturated cycloaliphatic residue optionally comprising at least one further heteroatom as a ring member, which cycloaliphatic residue may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent optionally may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
R³ denotes a linear or branched, unsaturated or saturated, unsubstituted or at least monosubstituted aliphatic residue;
R⁴ denotes hydrogen, a linear or branched, unsubstituted or at least monosubstituted, unsaturated or saturated aliphatic residue, an unsaturated or saturated, unsubstituted or at least monosubstituted, cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched alkylene group;
R⁵ denotes a linear or branched, unsubstituted or at least monosubstituted, unsaturated or saturated aliphatic residue, an unsaturated or saturated, unsubstituted or at least monosubstituted cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched C₁₋₃ alkylene group, or
R⁴ and R⁵ together with the nitrogen atom to which they are attached form a saturated or unsaturated cycloaliphatic residue optionally comprising at least one further heteroatom as a ring member, which cycloaliphatic residue optionally may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aliphatic residues may be substituted with substituents independently selected from the group consisting of halogen, hydroxy, —CN, —CF₃, —CHF₂, —CH₂F, C₁₋₆ alkoxy and optionally at least monosubstituted phenyl, the substituents of which may respectively be independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aryl residues may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅ -alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂ —NO₂, —CN, —CF₃, —CHF₂, —CH₂F, C₁₋₅ alkyl and C₁₋₅ alkoxy; and
wherein the above-stated cycloaliphatic residues may be substituted in the position of residues R⁴ and R⁵ with at least one substituent independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or substituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
in the form of a pure stereoisomer thereof or a mixture of stereoisomers in any mxing ratio, or a salt or solvate thereof.

26. A method according to claim 25, wherein 5-hydroxytryptamine uptake or noradrenalin uptake is inhibited.

27. A method according to claim 25, wherein μ opioid receptor activity is regulated.

28. A method of treating or inhibiting a disease or disorder at least partially mediated by a receptor selected from the group consisting of 5-hydroxytryptamine receptors, noradrenalin receptors and opioid receptors, or of effecting anxiolysis, diuresis, suppression of the urinary reflex, reduction of the addictive potential of opioids, modulation of locomotor activity, vasodialtion of arteries, or regulation of electrolyte balance in a patient, said method comprising administering to said patient a pharmaceutically effective amount of a substituted 2,5-diaminomethyl-1H-pyrrole compound corresponding to formula I: 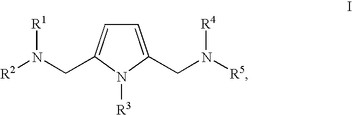
wherein
R¹ and R² together with the nitrogen atom to which they are attached form a saturated or unsaturated cycloaliphatic residue optionally comprising at least one further heteroatom as a ring member, which cycloaliphatic residue may be identically or differently mono- or polysubstituted with at least one substituent selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent optionally may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
R³ denotes a linear or branched, unsaturated or saturated, unsubstituted or at least monosubstituted aliphatic residue;
R⁴ denotes hydrogen, a linear or branched, unsubstituted or at least monosubstituted, unsaturated or saturated aliphatic residue, an unsaturated or saturated, unsubstituted or at least monosubstituted, cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched alkylene group;
R⁵ denotes a linear or branched, unsubstituted or at least monosubstituted, unsaturated or saturated aliphatic residue, an unsaturated or saturated, unsubstituted or at least monosubstituted cycloaliphatic residue optionally comprising at least one heteroatom as a ring member, which cycloaliphatic residue optionally may be attached via a linear or branched alkylene group, or an unsubstituted or at least monosubstituted aryl residue attached via a linear or branched C₁₋₃ alkylene group, or
R⁴ and R⁵ together with the nitrogen atom to which they are attached form a saturated or unsaturated cycloaliphatic residue optionally comprising at least one further heteroatom as a ring member, which cycloaliphatic residue optionally may be identically or differently mono- or polysubstituted with at least one substituent selected from the group consisting of C₁₋₅ alkyl, —C(═O)—O—C₁₋₅ -alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl; wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aliphatic residues may be substituted with substituents independently selected from the group consisting of halogen, hydroxy, —CN, —CF₃, —CHF₂, —CH₂F, C₁₋₆ alkoxy and optionally at least monosubstituted phenyl, the substituents of which may respectively be independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
wherein the above-stated aryl residues may be substituted with substituents independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or mono- or polysubstituted with at least one substituent independently selected from the group consisting of hydroxy, F, Cl, Br, I, —NH₂, —NH—C₁₋₅-alkyl, —N(C₁₋₅-alkyl)₂—NO₂, —CN, —CF₃, —CHF₂, —CH₂F, C₁₋₅ alkyl and C₁₋₅ alkoxy; and
wherein the above-stated cycloaliphatic residues may be substituted in the position of residues R⁴ and R⁵ with at least one substituent independently selected from the group consisting of C₁₋₅ alkyl, —C(═O)—O—C₁₋₅-alkyl, —O—C₁₋₅-alkyl, Cl, F, Br, I, —C(═O)—C₁₋₅-alkyl, —NH₂, —NH—C₁₋₅-alkyl, —N(C₁₋₅-alkyl)₂, optionally at least monosubstituted furanyl, optionally at least monosubstituted thiophenyl, optionally at least monosubstituted pyridinyl, optionally at least monosubstituted phenyl and optionally at least monosubstituted benzyl, wherein a furanyl, thiophenyl, pyridinyl, phenyl or benzyl substituent may itself be unsubstituted or substituted with at least one substituent independently selected from the group consisting of hydroxy, F, Cl, Br, I, —NH₂, —NH—C₁₋₅-alkyl, —N(C₁₋₅-alkyl)₂, —NO₂, —CN, —CF₃, —CHF₂, —CH₂F, C₁₋₅ alkyl and C₁₋₅ alkoxy;
in the form of a pure stereoisomer thereof or a mixture of stereoisomers in any mxing ratio, or a salt or solvate thereof.

29. A method according to claim 28, wherein said disease or disorder comprises pain.

30. A method according to claim 29, wherein said pain is selected from the group consisting of acute pain, chronic pain, and neuropathic pain.

31. A method according to claim 28, wherein said disease or disorder is selected from the group consisting of withdrawal symptoms, memory disorders, neurodegenerative diseases, epilepsy, cardiovascular disorders, water retention conditions, intestinal motility disorders, urinary incontinence, anorexia, tinnitus, pruritus, depression, sexual dysfunction, preferably erectile dysfunction, or airways diseases, and food intake disorders.

32. A method according to claim 32, wherein said disease or disorder is diarrhea; or erectile dysfunction; or a neurodegenerative disease selected from the group consisting of Parkinson's disease, Huntington's chorea, Alzheimer's disease and multiple sclerosis; or a food intake disorder selected from the group consisting of obesity, bulimia, anorexia, cachexia and non-insulin-dependent diabetes.
